(19) United States
(12) Patent Application Publication
(10) Pub. No.: US 2010/0062040 A1
(43) Pub. Date: Mar. 11, 2010

(54) WATER SOLUBLE DRUGS AND SUPPLEMENTS

(76) Inventors: Donald E. Ackley, Cardiff, AZ (US); Eugene Tu, San Diego, CA (US)

Correspondence Address: ROBERT A. PARSONS, 4000 N. CENTRAL AVENUE, SUITE 1220, PHOENIX, AZ, 85012, US

(21) Appl. No.: 12/545,650

(22) Filed: Aug. 21, 2009

Related U.S. Application Data
(60) Provisional application No. 61/090,982, filed on Aug. 22, 2008.

(51) Int. Cl. A61K 9/14; A61K 31/122; A61K 31/047; A61K 31/01; A61K 31/015; A61K 31/355; A61K 31/07; A61K 35/12; A61K 36/185; A61K 36/55; A61K 36/13; A61P 39/00

(52) U.S. Cl. 424/439; 424/94.1; 514/729; 514/691; 514/762; 514/763; 514/458; 514/725; 424/520; 424/725; 424/768; 424/776; 977/773

(57) ABSTRACT

A method of forming water soluble microparticles is disclosed that includes the steps of providing a water insoluble food supplement, mixing the food supplement in a water miscible polar solvent, heating the mixture to increase the solubility and dissolve the food supplement into the solvent to form a saturated solution, and streaming the solution into water to form a solvent-water mix such that microparticles of the food supplement are produced

### CROSS-REFERENCE TO RELATED APPLICATIONS

This application claims the benefit of U.S. Provisional Application No. 61/090,982, filed 22 Aug. 2008.

### FIELD OF THE INVENTION

This invention relates to structure and method for preparing water soluble microstructures.

### BACKGROUND OF THE INVENTION

At the present time energy drinks, functional waters and supplement drinks are very popular worldwide. The major problem is that many supplements are water insoluble which greatly reduces bioavailability, constrains delivery mechanisms, requires costly additives to increase solubility and/or reduces appeal to customers focused on organic products. This severely limits the opportunity to create new brands targeting lifestyles, younger demographics, etc.

The prior art generally includes liposome or polymer encapsulated drugs and further includes solid lipid nanoparticles. Oil soluble drugs and supplements suffer from poor bioavailability and can further suffer from delivery problems. Many prior art drinks use detergents or artificial coatings to get one or two antioxidants into water. Chemical modifications to the antioxidant molecular structure are also used. Prior art weight loss drinks are generally cloudy and sludgy with material at the bottom so that they must be agitated to mix the material. Many vitamins and supplements can only be provided in pill or powder forms and many have a bad taste or odor so that taking them is undesirable.

It would be highly advantageous, therefore, to remedy the foregoing and other deficiencies inherent in the prior art.

Accordingly, it is an object of the present invention to provide new and improved water soluble microparticles of a wide variety of drugs and supplements.

It is a further object of the present invention to provide new and improved water soluble microparticles of any of water insoluble antioxidants or vitamins, omega-3 fatty acids, drugs and other supplements.

It is another object of the present invention to provide new and improved water soluble microparticles of drugs and supplements that enable new product form factors such as anti-oxidant water and topical creams.

It is another object of the present invention to provide new and improved clear drinks including water soluble microparticles of drugs and/or supplements with no additives or colors and that taste and smell like water.

It is a further object of the present invention to provide desirable water drinks containing healthy oils and omega3 fatty acids at useful doses.

It is an object of the present invention to provide new and improved water soluble microparticles of a wide variety of drugs and supplements.

It is another object of the present invention to provide new and improved alcoholic drinks including water soluble microparticles of drugs and/or supplements with no additives or colors.

### SUMMARY OF THE INVENTION

Briefly, to achieve the desired objects of the instant invention in accordance with a preferred embodiment thereof, a method of forming water soluble microparticles is disclosed. The method includes providing a water insoluble food supplement, mixing the food supplement in a water miscible polar solvent, heating the mixture to a temperature as high as 70° C. to increase the solubility and dissolve the food supplement into the solvent to form a saturated solution, and streaming the solution into water to form a solvent-water mix such that self-organized microparticles of the food supplement are formed with diameters >100 nm.

In a specific embodiment of the present invention, water soluble microparticles are formed including a food supplement in which individual molecules have a chain-like structure with a more hydrophilic end and the soluble microparticle is arranged with the polar or hydrophilic ends at a surface of the microparticles for interacting with water.

In another embodiment of the present invention, a water soluble microparticle with an outer lipid membrane encapsulating one or more micro-formed food supplements is formed with surface receptors around the outer periphery.

In yet another embodiment of the present invention, water soluble nanoparticles of size <100 nm are formed.

### BRIEF DESCRIPTION OF THE DRAWINGS

The foregoing and further and more specific objects and advantages of the instant invention will become readily apparent to those skilled in the art from the following detailed description of a preferred embodiment thereof taken in conjunction with the drawings, in which:

FIG. 1 is a 400× photomicrograph of microparticles in solution;

FIG. 2 illustrates the dependence of particle size as a function of concentration, for Coenzyme Q10 in ethanol;

FIG. 3 is a schematic depiction of antioxidant microparticles;

FIG. 4 illustrates a typical light scattering size distribution of microparticles, with a size distribution peaked at 255 nm;

FIG. 5 is a schematic depiction of a solid lutein/CoQ10 combined microparticle;

FIG. 6 is an HPLC characterization of two runs in which statin and helper molecules are mixed;

FIG. 7 is a differential scanning calorimetry characterization illustrating the effects of different concentrations of statin molecules with helper molecules; and

FIG. 8 illustrates a microparticle formed in accordance with a secondary process.

### DETAILED DESCRIPTION OF THE DRAWINGS

In the new structure and method for preparing water soluble nanostructures from water insoluble food supplements or drugs, a key element is a process by which oil soluble supplements are dissolved in ethanol or another polar solvent often at elevated temperatures, typically at 50° C., to increase their solubility. The dissolved supplements are then combined with water or aqueous solutions at room temperature (20° C. to 25° C.) in ratios that range from 1:1 to 1:6 by volume. The resulting mixture is vigorously vortexed, and may be further combined with water to dilute the solution and achieve the smallest particle size and optimum monodispersity. The microstructures that result efficiently incorporate the supplements, with the supplement in the outer portion of the particle arranged into its most hydrophilic orientation. Lipids may be added to vary the particle size, surface energy, and bioavailability, and to provide linkers for targeting moieties.

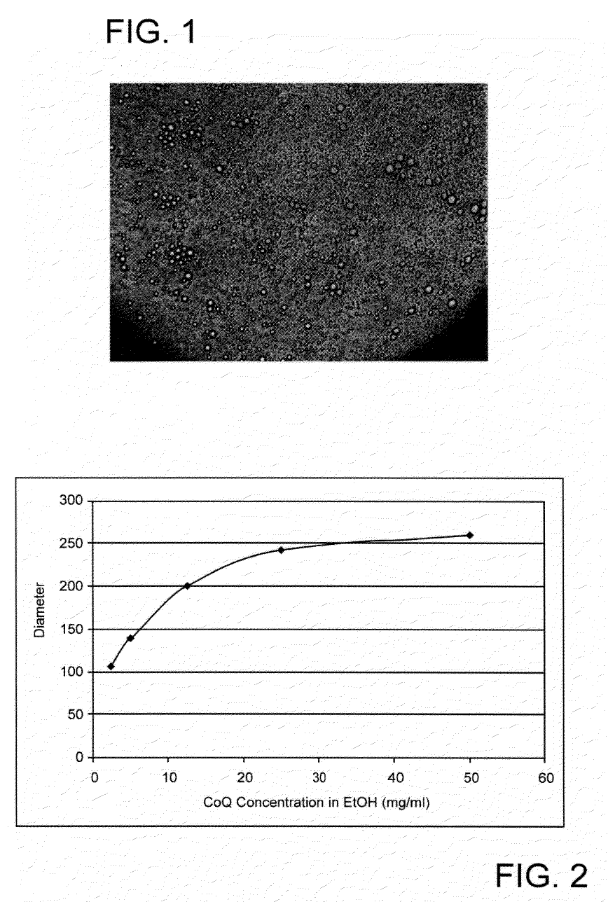

In a first embodiment, stable water soluble Coenzyme Q10 (CoQ) and other antioxidant, vitamin, carotenoid, healthy oil, or fatty acid microparticles are produced as a new form of the materials. To form CoQ microparticles, the material is dissolved in a water miscible polar solvent, such as ethanol, acetone, methanol, isopropanol, ethylene glycol, acetic acid, glycerol, and the like, or a mix thereof. Depending on the desired concentration of CoQ in the solvent, the solvent is heated to temperatures as high as 70° C. to increase the solubility of the CoQ. After the CoQ has dissolved into the solvent and formed a transparent (but yellow) solution, the solvent and CoQ are streamed (e.g. by using a pipettor) into water. As the CoQ is not soluble in the solvent-water mix, it self-organizes to form CoQ microparticles. For CoQ in ethanol, the water concentration can be as low as 20% and microparticle formation still occurs. A 400× photomicrograph of the particles in solution is shown in FIG. 1.

The microparticles remain stable in water, without dropping out of solution, for extended periods of time. The size may be varied by varying the concentration of the antioxidant in the solvent, as shown in FIG. 2 for CoQ microparticles formed using ethanol at room temperature as a solvent.

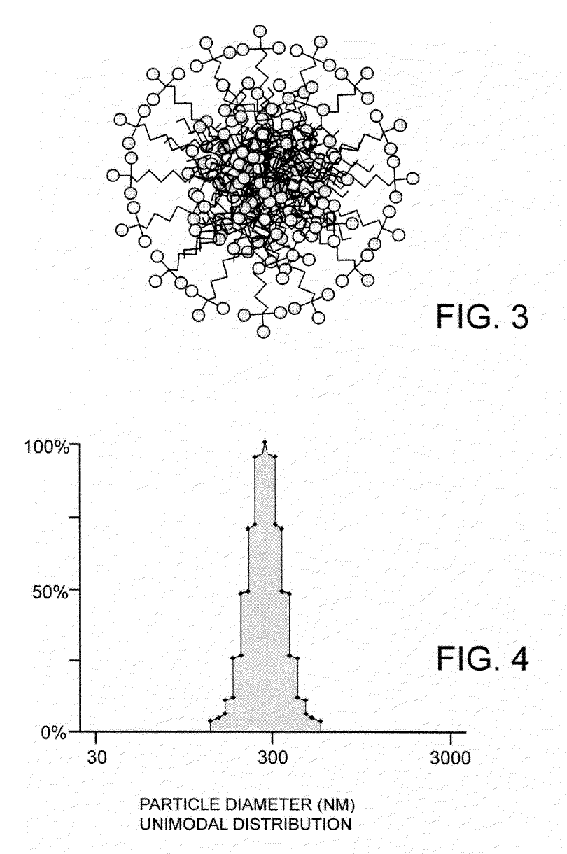

Microparticles have also been formed using the natural antioxidants lutein, zeaxanthin, lycopene, marigold extract, retinol palmitate (vitamin A), tocopherol (vitamin E), and beta carotene, as well as various oils and omega3 fatty acids. In order for the materials to form water soluble microparticles, the individual molecules must generally have a chain-like structure and align at the surface of the particles so that their polar or hydrophilic end is at the surface interacting with the water. An antioxidant microparticle is schematically depicted in FIG. 3. Some supplements may form nanoparticles with diameters <100 nm.

A second embodiment provides water soluble coenzyme Q (and other antioxidants) with lipids. Lipids may also be incorporated into the microparticles to alter their size, surface properties, or to add functional groups for targeting. To incorporate lipids, a solution of lipids in a water miscible solvent is added to the antioxidant solution before mixing with water to create the microparticles. Otherwise the process remains the same as described above. The lipid concentration is typically 2% to 20% by weight of the antioxidant concentration. Various lipids, including PC and PE, have been utilized in conjunction with the antioxidants. Using charged lipids such as DOTAP has the beneficial effect of preventing aggregation of the lipid containing microparticles. In addition to antioxidants, other materials of interest as food supplements have been encapsulated using this method, including fish oil. The microparticles that are formed are quite monodisperse, with diameters in the range of 100 nm to 500 nm. A typical light scattering size distribution, with size distribution peaked at 255 nm is illustrated in FIG. 4.

Either of the lipids or the payload supplement may be dissolved in a water immiscible (or partially miscible) solvent, as long as the mixture of the two forms a miscible mixture with water at the appropriate concentration. Small amounts of surfactants in the solvent phase may stabilize high concentrations of the oil-soluble supplements at low temperatures. Surfactants, however, are not necessary.

Oxidation of fragile payloads may be prevented during heating by providing an inert gas environment and minimizing the time at temperature.

The process may be run in batch mode, for example in a beaker or tank, by adding a stream of 50° C. CoQ in ethanol to a volume of water by stirring. The process may be inverted, beginning with a stirred volume of CoQ in ethanol at 50° C. and streaming water into it. There may be advantages to one process or the other in terms of particle size and composition. As an alternative to stirring, ultrasound may be used to enhance mixing.

Alternatively, the process may be run in continuous flow. In the continuous flow process the solvent/antioxidant flows in one tube (which can be heated as necessary) and the aqueous stream flows in another tube. The streams are combined in a T-junction or other mixing device and spontaneously form microparticles upon mixing. Active mixing may be incorporated into the mixing device. This process may have some advantages in that the reagents may be contained in temperature controlled tanks which can be pressurized with inert gases to maintain reagent quality and drive flow. Particle size is potentially controlled by varying the relative flow rates and the flow geometry, which may be important for enhancing bioavailability. The process is readily integrated and could be incorporated directly into a bottling line for drinks, for example. Microparticle formation in a continuous flow system of the sort described above has been successfully demonstrated.

Applications of the formulation technology include a wide variety of food supplements. Supplements demonstrated to date using the process include CoQ10, lutein, beta-carotene, marigold extract, vitamin E, retinol, tocopherol, coconut and other medium chain saturated oils, and omega3 fatty acids such as flaxseed oil, pine nut oil, borage oil, and fish oils. Given the small size and water stability of the microparticle formations, supplement drinks are of interest. Clear supplement drinks are of particular interest as the drinks will remain transparent at the usual concentration of supplements when they are solubilized using the disclosed process. A major advantage for the supplement market is that claims may be made as to natural or organic products, since the process can begin with natural supplements and by using an “organic” or non-GMO solvent such as ethanol fermented and distilled from organically grown grapes or corn.

Using the disclosed process, solutions of aqueous microparticles have been produced with as little as 25% water fraction. This results in a concentrated microparticle solution which may be suitable, with or without post processing, for delivery by means of conventional capsule formulations. As a capsule such as a SoftGel can tolerate 8% to 10% residual water content, it is possible to achieve a sufficiently low water content by a simple centrifugation process.

Concentrates with water fractions ranging from 25% to 75% may be prepared and packaged for later addition to water by a consumer. Residual polar solvent may be removed from the concentrates at any step in the process by well-known techniques of evaporation, rotary evaporation, solvent perfusion, and the like.

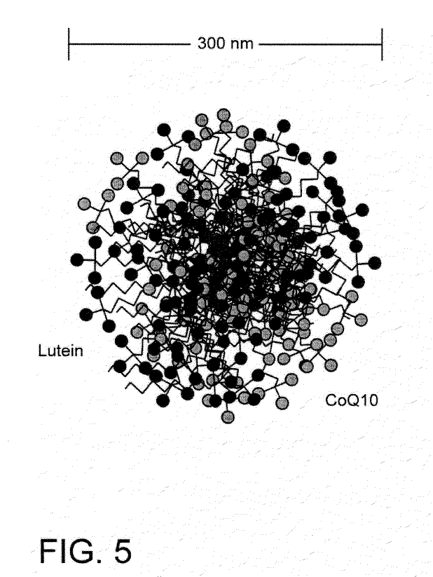

In addition, different supplements, such as lutein/CoQ10, lycopene/CoQ10, CoQ10/vitamin E, vitamin E/vitamin A, etc. may be combined synergistically into single microparticles. Referring specifically to FIG. 5, a schematic depiction of a lutein/CoQ10 combined microparticle is illustrated. In this example, the combined microparticle has a diameter approximately 300 nm. In this process the supplements are mixed prior to microparticle formation and subsequently form particles similar to that illustrated comprising both supplements. Combination particles may also include fat soluble food supplements that do not form microparticles in this process on their own. Examples of such supplements include resveratrol, curcumin, quercetin, plant phytosterols, and vitamin D.

An additional application is supplemented alcoholic drinks. Given the known benefits of ethanol in reducing cardiovascular problems, the addition of potent, encapsulated antioxidants such as CoQ or lutein may enhance the health benefits of moderate alcohol consumption. As the encapsulation process can utilize ethanol as an intrinsic part of the process, supplemented alcoholic drinks are a logical product. The supplements could be readily synthesized or merely added at the blending step for the alcoholic beverage. As the microparticles are routinely made at ethanol concentrations of commercial interest, the microparticles will be stable in the alcoholic blends. Stability over months has been observed in samples that had roughly 50:50 mixes of ethanol and water.

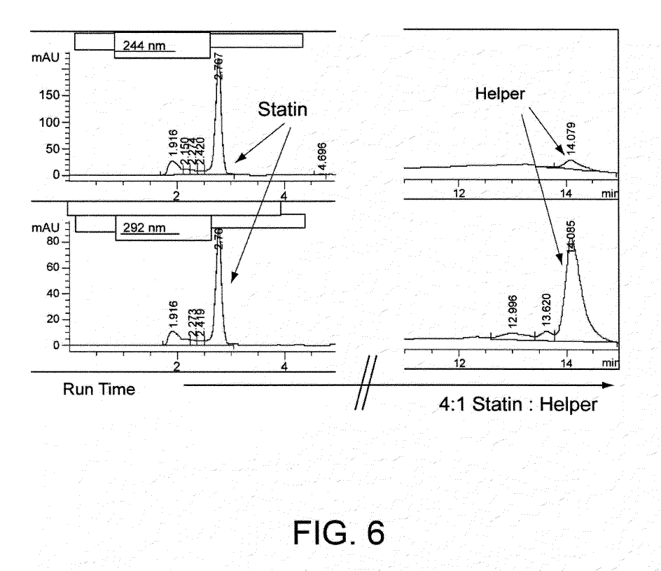

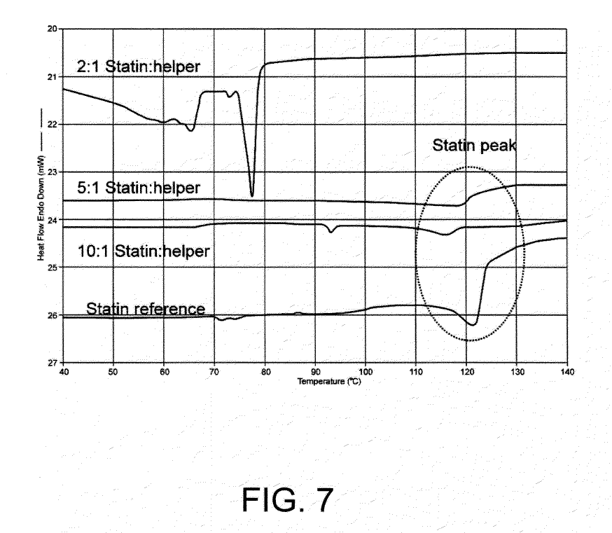

The present process should also be of particular interest for encapsulating oil soluble drugs with structures that resemble carotenoids. For example, retinol has orphan drug status for some indications. Some other drugs that might make microparticles based on their similarity to lipids are phytosterols, fibrates, lipstatin (Orlistat), and polyene antifungals. It may also be possible to use the antioxidants as stabilizers or helpers for other water insoluble drugs such as proteins or peptides. Turning to FIG. 6, two runs in which statin and helper molecules are mixed show efficient incorporation of the statin molecules with the helper molecules. Also, FIG. 7 illustrates the effects of different concentrations of statin molecules with the helper molecules.

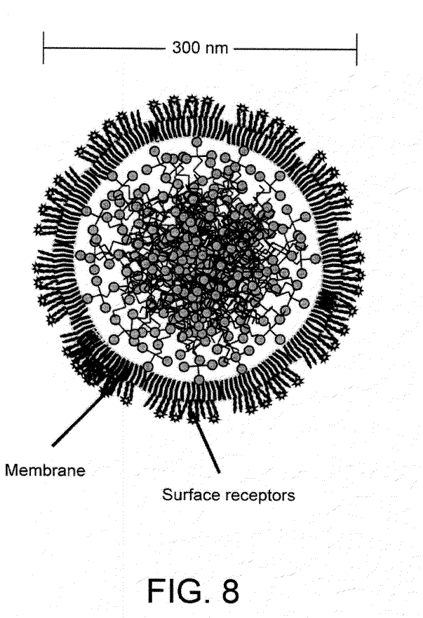

Turning to FIG. 8, a microparticle formed in accordance with a secondary process is illustrated. In this microparticle, an outer lipid membrane is formed to encapsulate micro-formed supplements, for example vitamin E, with surface receptors around the outer periphery. The microparticle illustrated has a diameter of approximately 300 nm. The process can also be used to wrap membranes around larger (e.g. 1 um to 15 um) structures to alter their surface properties or to contain liquid trapped in the hollow structure. This process is also suitable for supplements that are presented in solid, non-soluble forms.

Thus, new and improved water soluble microparticles of drugs and supplements and methods of production are disclosed. Also, the new and improved water soluble microparticles of drugs and supplements enable new product form factors such as anti-oxidant water and topical creams. The advantages of the new and improved water soluble microparticles include significantly enhanced water solubility, monodisperse size distribution, good short and long-term stability, the ability to incorporate targeting moieties, and a synthesis process that is simple, low-cost, and efficiently utilizes the supplement payload. Also, using the improved methods water soluble microparticles can be provided to produce clear antioxidant waters, natural supplement weight loss drinks, omega-3 drinks, and the like which have not been previously available.

Various changes and modifications to the embodiments herein chosen for purposes of illustration will readily occur to those skilled in the art. To the extent that such modifications and variations do not depart from the spirit of the invention, they are intended to be included within the scope thereof which is assessed only by a fair interpretation of the following claims.

Having fully described the invention in such clear and concise terms as to enable those skilled in the art to understand and practice the same, the invention claimed is:

1. A method of forming water soluble microparticles comprising the steps of:
providing a water insoluble food supplement;
dissolving the food supplement in a water miscible polar solvent;
mixing the solution into water to form a solvent-water mix so as to create microparticles of the food supplement.

2. The method of claim 1 wherein the food supplement forms nanoparticles with diameter <100 nm.

3. A method as claimed in claim 1 wherein the step of providing the food supplement includes providing at least one of CoQ10, lutein, zeaxanthin, fucoxanthin, astaxanthin, lycopene, beta-carotene, resveratrol, vitamin E, retinol, coconut oil, flaxseed oil, borage oil, black currant oil, pine nut oil and fish oils.

4. A method as claimed in claim 1 wherein the step of
dissolving the food supplement in the water miscible polar solvent includes mixing in one of ethanol, acetone, methanol, isopropanol, ethylene glycol, acetic acid, and glycerol.

5. A method as claimed in claim 1 wherein the step of mixing the solution into water includes mixing into water at room temperature (20° C. to 25° C.) in ratios that range from 1:1 to 1:6 by volume.

6. A method as claimed in claim 1 further including a step of heating the polar solvent to increase the supplement solubility.

7. A method as claimed in claim 1 further including a step of vigorously mixing the solution to achieve the smallest particle size and optimum monodispersity.

8. A method as claimed in claim 1 further including a step of adding lipids for varying one of particle size, surface energy, and bioavailability.

9. A method of forming water soluble microparticles comprising the steps of:
providing a water insoluble supplement in which individual molecules have a chain-like structure with a polar or hydrophilic end;
dissolving the antioxidant in a water miscible polar solvent;
mixing the solution into water to make a solvent-water mix so as to form microparticles of the antioxidant, each of the microparticles arranged with the polar or hydrophilic ends at a surface of the microparticles interacting with the water.

10. The method of claim 9 wherein the step of providing a water insoluble antioxidant includes providing at least one of coenzyme Q10, lutein, zeaxanthin, astaxanthin, marigold extract, fucoxanthin, lycopene, retinol, retinol palmitate (vitamin A), tocopherol (vitamin E), and beta carotene.

11. A method as claimed in claim 9 wherein the step of dissolving the antioxidant in the water miscible polar solvent includes mixing in one of ethanol, acetone, methanol, isopropanol, butyl alcohol, acetic acid, ethylene glycol, and glycerol.

12. A method as claimed in claim 9 wherein the step of mixing the solution into water includes mixing into water at room temperature (20° C. to 25° C.) in ratios that range from 1:1 to 1:6 by volume.

13. A method as claimed in claim 9 further including a step of vigorously mixing the solution to achieve the smallest particle size and optimum monodispersity.

14. A method as claimed in claim 9 further including a step of adding lipids for varying one of particle size, surface energy, and bioavailability.

15. A water soluble microparticle with an outer lipid membrane encapsulating one or more micro-formed food supplements with surface receptors around the outer periphery.

16. A water soluble microparticle as claimed in claim wherein the one or more micro-formed food supplements include at least one of CoQ10, lutein, beta-carotene, resveratrol, tocopherol (vitamin E), retinol, retinol palmitate (vitamin A), and fish oils.

17. A water soluble microparticle including at least one food supplement in which individual molecules have a chain-like structure with a polar or hydrophilic end and the soluble microparticle is arranged with the hydrophilic ends at a surface of the microparticles for interacting with water.

18. The water soluble microparticle as claimed in claim 17 wherein the food supplement includes at least one water insoluble antioxidant selected from at least one of coenzyme Q10, lutein, zeaxanthin, astaxanthin, marigold extract, lycopene, retinol palmitate (vitamin A), tocopherol (vitamin E), and beta carotene.

19. The water soluble microparticle as claimed in claim 17 wherein the food supplement includes a water insoluble omega 3 fatty acid selected from one of flaxseed oil, coconut oil, pine nut oil, borage oil, black currant oil, or fish oils.

20. The water soluble microparticle as claimed in claim 17 wherein the food supplement is combined with a fat soluble molecule lacking the chain-like structure so as to form a water soluble combination particle.

21. The water soluble combination particle of claim 20 wherein the fat soluble molecule is chosen from one of resveratrol, quercetin, curcumin, Vitamin D, plant phytosterols, or statins.
